(19) United States
(12) Patent Application Publication
(10) Pub. No.: US 2010/0145342 A1
(43) Pub. Date: Jun. 10, 2010

(54) DISPOSABLE ACETABULAR REAMER AND METHOD OF FABRICATING THE SAME

(76) Inventors: Richard L. Grace, Dover, AR (US); Christopher R. Grace, Russellville, AR (US); Louis Paul Chalfant, JR., Hot Springs, AR (US); Douglas Michael Dawson, Little Rock, AR (US)

Correspondence Address: LADAS & PARRY LLP, 224 SOUTH MICHIGAN AVENUE, SUITE 1600, CHICAGO, IL, 60604, US

(21) Appl. No.: 12/280,199

(22) PCT Filed: Feb. 22, 2006
(86) PCT No.: PCT/US06/06112

§ 371 Date: Aug. 21, 2008

(51) Int. Cl. A61B 17/00; B21K 5/04

(52) U.S. Cl. 606/81; 606/79; 76/115

(57) ABSTRACT

A disposable acetabular reamer having a cutter blade (16) molded into the surface of a support (11). The cutter blade is formed of a single flat sheet of metallic material having a series of teeth (24) formed thereon which protrude above the surface of the support such that when the acetabular reamer is rotated, the teeth contact and reduce the surface of a boney socket. The invention includes a novel method of forming the reamer which includes shaping the cutter blade once teeth are formed therein and integrating the cutter blade into the support coincident with the shaping process. The assembled unit is so economical that it may be discarded after use.

The present invention relates generally to acetabular reamers used in various orthopedic procedures and, more particularly, to acetabular reamers, including the cutter blade, support, and a novel method of fabricating the same, which results in an exceptionally high quality medical instrument, capable of being made so efficiently, relative to other competitive instruments, that it may be economically disposed of after a single use.

### BACKGROUND OF THE INVENTION

1. Field of the Invention

The acetabular reamer is a surgical instrument for preparing a socket in the pelvis bone for receipt of an artificial prosthesis. In use for many years, such devices are typically bowl-shaped and mounted on a centrally disposed shaft which is capable of being rotated by, for example, an electric drill. Such reamers must be capable of creating smooth cavities in bone to very exacting tolerances and must, therefore, be exceedingly sharp, easily manipulated at the end of a driver shaft and be capable of readily disposing of boney particulate material created during use.

Medical devices are notoriously expensive, and, where they are invasive in nature, their repeated use requires rigorous measures to ensure their sterility in preparation for each subsequent use. Given this environment, work has been directed to making disposable instruments which are also capable of being cost effective. Such a disposable device is represented by this application.

2. Overview of the Related Art

Paul Salyer is recognized in this field as having several patents directed to acetabular reamers, including U.S. Pat. No. 5,100,267, for what is referred to as a “disposable” acetabular reamer. The cup, which is the working part of the instrument, is hemispherical in shape with cutting edges stamped on its adjacent perforations in accordance with his earlier U.S. Pat. No. 4,811,632.

Salyer also developed a patella cutter, which is taught in U.S. Pat. No. 5,299,893, which is a continuation-in-part of his now issued '267 patent. The patella cutter includes a flat, disk-like cutter lid having cutting teeth thereon and is supported on a bowl which is of a plastic material.

Salyer, as previously stated, has been prolific in his development of various reamer configurations and drivers therefor. His U.S. Pat. No. 6,428,543 is a recent effort which discloses a process for manufacturing an acetabular reamer which is representative of the current thinking among those skilled in the art in the manufacture of such devices. Salyer teaches the initial formation of a bowl 48 by a deep draw process which results in considerable metal stress. Thereafter, Salyer forms a plurality of holes about the bowl in a spiral pattern. A cutting portion is formed on the margin of the hole and then deformed outwardly so as to be raised above the outer margin of the bowl.

Da Rold, in his U.S. Pat. No. 5,968,049, proposes a three step operation. The first step, as in Salyer, is to form the cup and, thereafter, the cutting edges, and, finally, to raise the cutting edges above the surface of the cup. Kudla U.S. Pat. No. 5,203,653 reflects a process whereby the cutting surfaces are formed at the edges of arcuate slots in a hemispherical blank.

In Salyer, Da Rold and Kudla, as in prior art methods for formation of a reamer, the process for accurate formation of holes and cutting edges in the cup is both arduous and expensive, each such opening being necessarily formed individually in a hemispherical blank.

Not all reamers are hemispherical. For example, Frieze, in his U.S. Pat. No. 5,755,719, suggests a different approach to that of the cup. Frieze proposes a number of arcuate, intersecting cutting blades. He does not suggest, or purport to provide, a disposable acetabular reamer.

Prizzi, Jr. U.S. Pat. No. 5,514,141 departs from the more conventional cup configuration in favor of a series of upwardly and outwardly extending flutes having cutting edges.

It will become apparent from a brief overview of the existing art that far more attention has been paid to the end result than how it is arrived at. By way of example, the cutter element on modern day reamers is formed by creating a series of teeth in a strip of material and then forming the strip into the desired shape. The novel process of the present invention is the virtual antithesis of the currently accepted process.

As will be apparent from a review of the existing art, in comparison with the features of the present invention, those features result in a disposable acetabular reamer which is both novel and utilitarian.

### SUMMARY OF THE INVENTION

It is a primary objective of the present invention to provide an acetabular reamer for the medical community, as a highly useful instrument, which has distinct advantages over the existing state of the art.

More particularly, it is an objective of the present invention to provide a medical instrument in the form of an acetabular reamer which is fabricated by a novel process, which permits such economical construction that it is both exceptionally sharp, efficient in assembly and use, while being so cost effective as to be selectively disposable after a single use.

It is a specific purpose of the present invention to provide the medical profession with a distinct advantage by creating a cutter blade which is initially formed into its requisite shape and, thereafter, formed with cutting edges in the form of teeth. In so doing, it provides a cutter which is precisely formed to present the ideal surface to shape the boney material it must shape, with the cutting surfaces presenting optimum shaping capability, in an exceptionally economical manner.

Yet another objective of the present invention is to provide an instrument which permits an improved procedure in which use of the instrument, by virtue of its size and configuration, is less invasive than more conventional instruments.

It is an additional, and further, objective of the present invention to provide an improved disposable acetabular reamer which is exceptionally easy to control in use, and to remove, in a most efficient manner, only the precise material to be removed, with great accuracy.

The foregoing, as well as other objects and advantages of the present invention, will become apparent to those skilled in the art upon a reading of the accompanying Detailed Description of a Preferred Embodiment, when taken in conjunction with the drawings, wherein:

### BRIEF DESCRIPTION OF THE DRAWINGS

FIG. 1 is a pictorial representation of the reamer as it would appear in the environment in which it has particular use, with the stem, or shaft portion 17, of the driver to emphasize the structure of the reamer itself;

FIG. 2 is a further view of the reamer of the present invention, illustrating the cutting surfaces in considerable detail;

FIG. 3 is a plan view of the cutter blade constructed in accordance with the present invention;

FIG. 4 is an exploded view of an acetabular reamer, including at least a significant portion of the driver/support assembly;

FIG. 5 is an alternative, and much thinner, cutter blade, shown in top plan view;

FIG. 5A is a view similar in nature to FIG. 3, illustrating a support which is formed integrally with the cutter blade of FIG. 5, which is circumscribed by the plastic shell defining the driver superstructure;

FIG. 6 is yet another blade configuration in which the blade profile resembles a wagon wheel;

FIG. 7 is an alternative blade configuration in which the blade profile comprises a series of blades fanning outwardly from a central focal point; and

FIG. 8 is yet another modified form in which outwardly reaching, longitudinally extending, opposed legs provide the cutting surfaces.

### DETAILED DESCRIPTION OF A PREFERRED EMBODIMENT

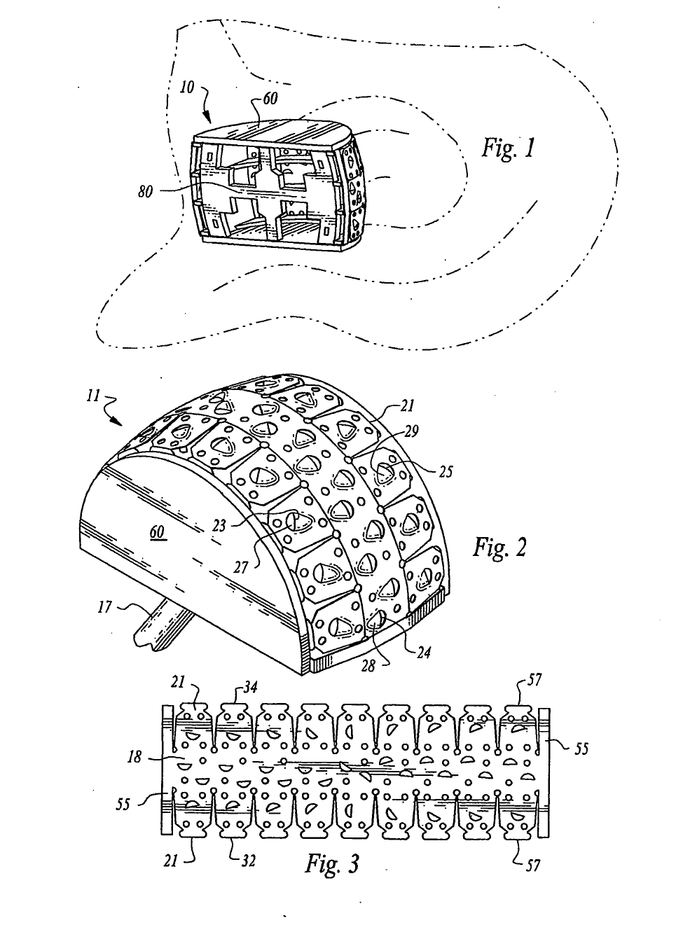

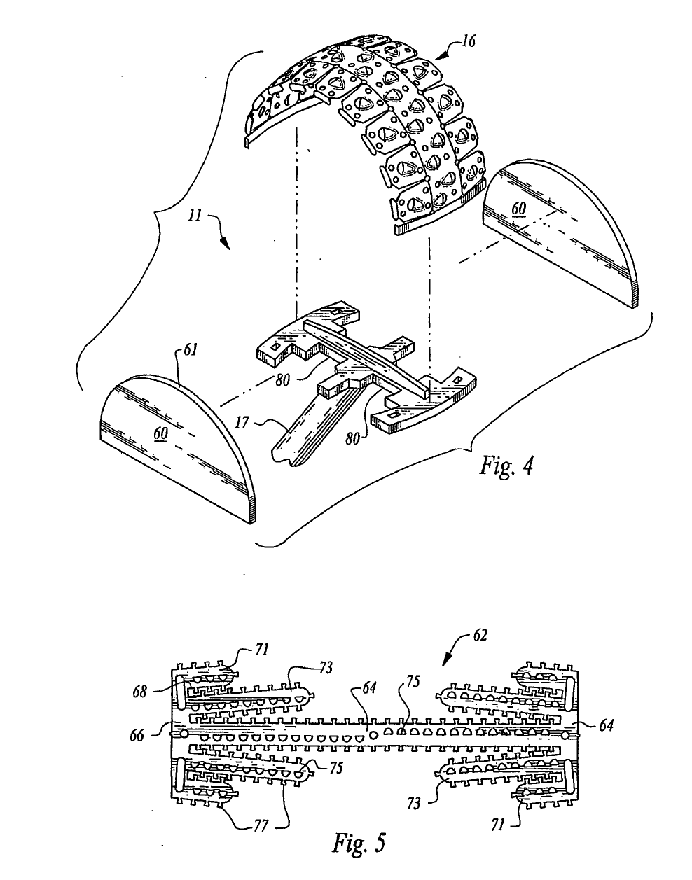

With reference now to the drawings, and initially to FIG. 1, an acetabular reamer 10, fabricated in accordance with the present invention, is illustrated pictorially. The reamer is of a generally hemispherical shape, which is fashioned to conform to the shape of a hip prosthesis and is intended to prepare and refine the interior surface of the pelvic socket for receipt of a prosthesis.

For purposes of clarity, the term “generally” hemispherical is chosen to indicate that while segments of the cup may be removed, or deleted, as it would appear in its static orientation, when rotated on its longitudinal axis, it cuts, or reams, a generally hemispherical swath.

The reamer 10 comprises, in its essential parts, a working shell, or cup-like, support assembly 11. The driver and shell combine with the cutter blade 16, properly positioned and nested on the arcuate surface of the cup, to define the shell, or cup-like, support assembly 11. Rotation of the shell assembly on its longitudinal axis, cuts, or reams, a generally hemispherical swath in the socket of the hip bone by removing, macroscopically, precise amounts of boney material, until the proper cavity is achieved.

The support assembly 11 is rotated about the longitudinal axis of a shaft 17, secured to the support assembly, and which is driven by a suitable power source, e.g., an electric drill (not shown). The cutter blade, which is nested on the support is, thus, rotated within the boney socket, in a hemispherical path to remove a controlled amount of material therefrom.

In previous devices, the cup-like shape is achieved through a deep-drawing process. Because of the severe deformation that occurs, the metal blank must have sufficient wall thickness and be sufficiently soft to permit the drawing process. In certain areas of the cutter blade, considerable thinning of the wall thickness occurs and considerable stress is imparted into the metallic blank. The stresses are so great that any irregularity in the stress distribution caused by abrupt changes in form, such as holes, scratches, or inclusions in the metal will cause stress concentrations, possibly leading to the formation of cracks, and tears in the metal blank, rendering the metal blank useless. For this reason, the blank must be free from holes during the drawing process and then the cutting teeth must be formed, raised, and sharpened individually, and the now three-dimensional shape heat-treated so that the device can effectively cut hard bone.

The present invention, by contrast, employs a novel approach whereby the cutting teeth may be formed, sharpened, and raised from a previously hardened flat blank prior to being transformed into a generally hemispherical shape, enabling considerable savings to be achieved without detriment to the desirable characteristics of accuracy, efficiency, sharpness and ease of use.

The process of forming the cutter blade, and ultimately the reamer of the present invention, entails several, heretofore unrecognized steps, some of which may be performed simultaneously. For clarity, they will be developed individually. As a first step in the process, the desired profile of the cutter blade 16 is established, and, in the accompanying drawings, it is rectangular. The profile may be economically formed from a single blank of thin metallic material by a stamping procedure, although it will be understood that it may be formed of several discrete elements making up the cutter blade, where appropriate, and may also be produced from other materials, e.g., ceramic. Indeed, any medically acceptable material which is also capable of providing a sharp edge is within the contemplation of the invention.

In one of its preferred forms, illustrated in FIG. 3, the cutter blade 16 consists of a generally rectangular center section 18, flanked on either side by serially spaced segments in the nature of wings 21, extending outwardly transverse to the longitudinal axis of the center section. As will be seen directly, an end portion of the segments 21 also serve as anchor arms to secure the cutter blade in the proper position on the support assembly 11.

In addition to defining the center section 18 and the wings 21, the same first step of the process creates a series of rows of cutting edges, or teeth, 23, 24 and 25, both on the wings 21 and the center section. The specific orientation and number of teeth may vary, especially as the size of the reamer varies, while accomplishing the objective of making sure complete and symmetrical reduction of the joint socket is achieved. That occurs when the entire surface to be treated by the reamer is completely exposed to at least some of the teeth, as the reamer is rotated, to thereby remove any score lines, or roughness, that might otherwise occur if a portion of the surface is not contacted by at least one tooth as the reamer is rotated in the socket. To this end, there is at least one cutting edge, in the plane of the cutter blade, facing in a direction which is essentially transverse to the direction of rotation of the reamer, such that the cumulative effect is to have cuffing edges facing in all directions throughout a full 360 E of rotation of the cup 11.

In keeping with the objective of the invention to efficiently remove particulate created by the cutting process, the FIG. 3 embodiment includes openings 27, 28 and 29, respectively. In at least one process for forming the cutter blades, the openings may be simultaneously created immediately below, and coincident with, the cutting teeth. Thus, as boney material is removed by the cutting teeth, it is removed from the socket and migrates gravitationally through the openings 27, 28 and 29 and through the reamer to a collection area remote from the bone.

The transformation of a planer metal blank into a viable, exceptionally sharp, cutter blade having a distinctive profile, is efficiently accomplished by means of the single sided photochemical etching process, which is the subject of U.S. Pat. No. 5,100,506 to Sturtevant et al. It will be understood, however, that alternative methods of producing the same result with, perhaps, some increase in cost and somewhat less efficiency, are nonetheless within the contemplation of the invention.

Referring to FIG. 3, the configuration of the cutter blade 16 is to be formed, and, having established the desired profile of the flat blank as an initial step of the process, the cutter blade is configured. Once the blank profile is established, the next step is to subject the blank to a photo chemical etching process whereby the cutting edges, or teeth, are simultaneously formed to a sharpened condition and several relief openings, immediately adjacent the teeth, are strategically positioned. It will be understood that establishment of a profile and the etching of the teeth may be accomplished by the same photo chemical process.

In a next step, the teeth are then raised to a predetermined position above the surface of the cutter blade in order that they may make contact with the surface of the socket to be prepared by the reamer. This is accomplished by simply deforming the edges of the openings, by the use of an appropriate die, simultaneously, upwardly, while the cutter blade is still in plane as an otherwise flat sheet of material.

It will be appreciated that, as a practical matter and as a cost saving, several cutter blade blanks may be efficiently formed from a single sheet of material, without departure from the invention.

The resultant configurations can be observed in FIGS. 3, 5, 7 and 8. The tooth structure of the cutter blade is formed in keeping with the objective of providing total exposure of the surface of the socket to be finished, to cutting teeth. To this end, it will be seen that the illustrated pattern (FIG. 3) of cutting teeth 24, first on the left flank of the center section 18, are uniformly spaced along a generally elongated row, facing initially outwardly toward the outer edge 32 of each respective wing 21 and are rotated approximately 180 E as one views them moving from left to right as seen in FIG. 3.

In similar fashion, the companion cutter teeth 24, seen on the right flank of the center section, are formed so as to be rotated through an approximate 180 E, beginning by facing generally outwardly toward the outer edge tab 34 of each of the wings on the right flank of the center section 18.

Cutting teeth 23 and 25 are formed on the wings 21 and are similarly oriented in order that the objective of creating full contact with the socket to be treated can be more readily achieved. It will be appreciated that the specific orientation, size and number of the cutter teeth may be varied, depending on the specific task to be performed and the direction of rotation to be used, all without departure from the essential features of the invention.

In a final step in the reamer fabrication, the blade is shaped into a generally arcuate configuration by attaching said blade to the surface of the support, which is specifically configured to conform to the socket of the boney material to be dressed. This is accomplished, in accordance with the broadest terms of the present invention, by placing the previously configured, planer blank, into an appropriate forming device and joining the blank with the support structure.

In keeping with the objective of building a reamer that will have all of the characteristics which the medical profession needs, while maintaining cost effectiveness, the cup-like support assembly 11 comprises a pair of spaced arcuate side panels 60, having arcuate surfaces 61. In order to provide adequate support for the cutter blade without permitting deformation during use, the side panels are readily formed of a suitable, medically acceptable, material such as stainless steel. It will now be appreciated that the resultant reamer assembly, when rotated about its longitudinal axis, will cut a generally hemispherical shaped swath.

The cutter blade and support structure may be joined, with the cutter blade resting on the surface 61 thereof, and the wings bent into contact with the end plates 60, a base member 80, secures the end plates 60 in parallel spaced relation, by various methods known by one skilled in the art. Examples include, but are not limited to, welding, plastic injection molding, brazing, rivets, screws, other fastening devices, etc. A preferred method of joinder is facilitated by the formation of the tabs 57 about the perimeter of the blank. To effect joinder, an additional forming operation may be employed to assist the joining of the blade and support structure. In the present invention, to effect joinder, tabs 57 are bent to a proper angle so that they may be readily joined to the support structure. It may be appreciated that the raising of the cutting edges and the forming of the tabs 57 may be performed simultaneously, or in any order.

A word of caution. In order that the raised cutting edges of the cutter blade are not compressed, or deformed, in any way from their previously raised position accomplished by the previous step, care must be taken during the joining process to apply pressure only to areas of the cutter blade free from the raised cutting edges.

The support structure joined to the cutter blank must be sufficiently rigid to contain the cutter blank as it makes contact with the boney material. The support structure consists of two semi-circular plates that flat edge having been bent at a near 90″ angle to form a flange. The flange may now be easily attached to the driving (rotating) mechanism. Alignment holes have been provided in the flanges to allow for proper alignment and a sturdy connection to the driver device.

With reference now to FIG. 5, a somewhat different, but equally effective, blade configuration is illustrated, although the same fabrication process is applicable.

Thus, a blade 62 has a relatively narrow center section 64 terminating in somewhat wider transverse end strips 66. Fanning inwardly from the inner edge 68 of each of the transverse end strips 66 are a series of fingers 71 and 73, the latter being somewhat longer than the former.

As in the case of the cutter blade 16, cutter teeth 75 are etched into the blade 62 and a plurality of tabs 77 are formed on the fingers and the center section to permit the blade to be molded about, in accordance with the method of the present invention, integrally with a base member 80.

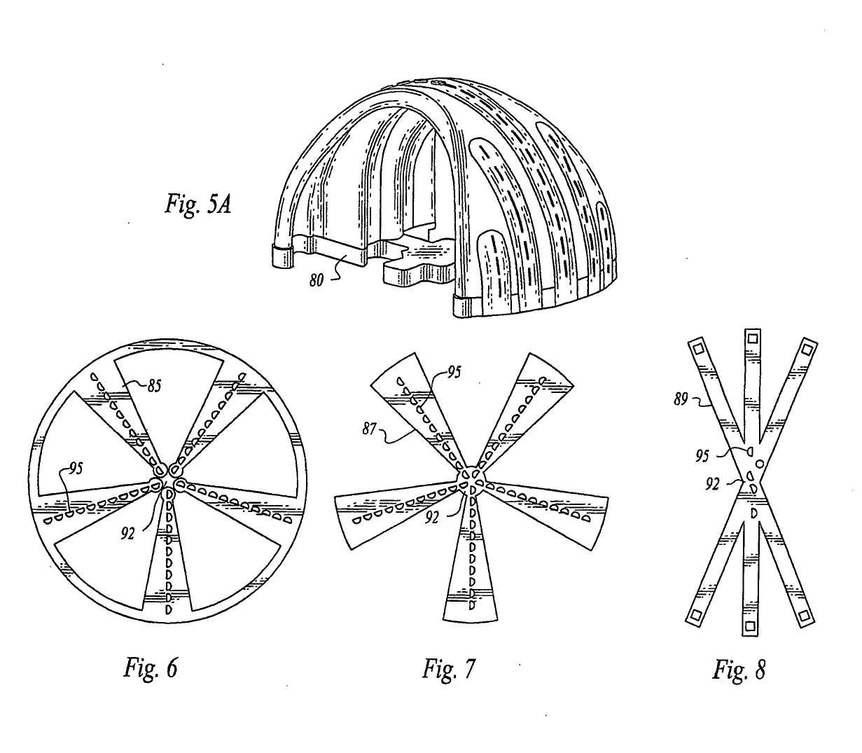

Referring now to FIGS. 6, 7 and 8, the versatility of the cutter blade of the present invention will become evident to those skilled in the art by observation of the variety of blade profiles possible in the practice of the method of the present invention. Those figures illustrate three diverse configurations in which outwardly extending segments in the nature of blade members 85, 87 and 89 fan outwardly from a central section, in the nature of a focal point 92, with cutting edges, or teeth, 95 formed strategically thereon. Each of the FIGS. 6, 7 and 8 may then be formed integrally with a support, as defined by the present method, to create the cup of the present invention.

This particular configuration is adaptable to an exceptionally thin cup formed of an equally narrow matrix with opposed arcuate segments removed such that teeth peak at the middle of the center section 92. The resultant unit is overall much more compact and more maneuverable in tight quarters, and further illustrates, by example, the versatility of the present invention.

It will be evident to one skilled in the art that some variation in the specific details of the various components of the disposable reamer described here are well within the contemplation of the invention as claimed.

What is claimed is:

1. A cutter blade formed from a flat sheet of material for use as an element of an acetabular reamer, said cutter blade being formed by means of the steps of:
a. forming a cutter blade profile;
b. forming a series of cutting edges on said cutter blade profile;
c. forming openings immediately adjacent each cutting edge;
d. deforming said cutting teeth so as to protrude above the plane of said cutter blade; and thereafter;
e. placing said cutter blank on an arcuate support member, and securing said cutter blade on the surface of said arcuate support member; creating a generally hemispherical shape.

2. The cutter blade of claim 1, wherein attachment tabs on said blade blank may be formed to facilitate attaching said blade blank to said support.

3. The cutter blade of claim 1, wherein steps a, b and c are performed simultaneously.

4. The cutter blade of claim 1, wherein steps a, b and c are performed by a photochemical etching process.

5. The cutter blade of claim 1, wherein steps a, b and c are formed by a single sided photochemical etching process.

6. The cutter blade of claim 1, wherein said cutter blade profile comprises a series of outwardly extending blade members, at least some of said blades having cutting edges formed thereon.

7. The cutter blade of claim 1, wherein said cutter blade profile comprises an elongated center section, a plurality of wings extending outwardly in said center section, said wings being generally transverse to the longitudinal axis of said center section.

8. The cutter blade of claim 1, wherein said cutter blade profile is in the form of a wagon wheel, which comprise a series of segments extending outwardly from a center section, terminating in a rim circumscribing said segments; said segments being formed with cutting teeth thereon.

9. The cutter blade of claim 1, wherein said cutter blade profile is in the form of a center section; segments extending outwardly from said center section in a divergent manner; and teeth being formed on said segments.

10. The cutter blade of claim 6, wherein steps a, b and c of claim 1 are performed simultaneously.

11. The cutter blade of claim 6, wherein steps a, b and c are formed by a photochemical etching process.

12. The cutter blade of claim 6, wherein steps a, b and c are formed by a single sided photochemical etching process.

13. A disposable acetabular reamer for preparing a socket in boney material for receipt of an artificial joint or the like, comprising a cutter blade and a support;
said cutter blade being formed from a blank of flat material to provide a predetermined profile, including a center section, a series of opposed segments extending outwardly from said center section;
said cutter blade being formed with a plurality of cutting edge, openings in said blank immediately below each said cutting edge to permit discharge of material being removed from said boney material; and
said support having a generally hemispherical shape; said support including a base plate, a slot depending from the longitudinal axis of said support; said cutter blade being formed about and secured on said hemispherical surface on said support; said support being shaped to conform to the socket;
said support being rotatable to cut a swath to reduce and refine the socket in said boney material when it is rotated about its longitudinal axis.

14. The disposable acetabular reamer of claim 13, wherein said cutter blade is formed in said support to secure said cutter blade against inadvertent movement when said reamer is in use.

15. The method of fabricating a cutter blade for use as an element of an acetabular reamer, comprising the steps of:
a. forming a cutter blade profile from a flat planer piece of material;
b. forming a series of cutting edges on said cutter blade profile;
c. forming openings immediately adjacent each cutting edge;
d. deforming said cutting teeth so as to protrude above the plane of said cutter blade;
e. attaching said cutter blade to an arcuate support to form a generally hemispherical shape.

16. The disposable acetabular reamer of claim 13, wherein said teeth formed on said cutter blade in which said cutting edges are so oriented as to provide full contact with the socket to be reduced by said reamer as said reamer is rotated.

17. The disposable acetabular reamer of claim 13, wherein each of said opposing segments terminate in at least one tab;
said tabs being fitted into said support to secure said cutter blade thereto.

18. A disposable acetabular reamer comprised of at least one cutter blade and a support;
said cutter blade and said support being affixed to one another such that said cutter blade is fixed in a generally arcuate configuration along the surface of said support;
a plurality of upstanding teeth formed on said cutter blade, and said cutter blade being capable of engaging the surface of a socket similarly shaped to thereby reform the surface of the socket.

19. The disposable acetabular reamer of claim 18, wherein said cutter blade is placed into a mold, said mold having a generally hemispherical cavity;
plastic material being introduced in said mold to form said support, and in the same process, shape and affix said cutter blade to the surface thereof.

20. The disposable acetabular reamer of claim 18, wherein said cutter blade is provided with upstanding sharpened edges formed therein by a photochemical etching process.

21. The disposable acetabular reamer of claim 15, wherein said support includes end plates, said tabs being fitted on said end plates to secure said cutter blade on said support.

22. A cutter blade for use as an element of an acetabular reamer, said cutter blade being formed from a blank to provide a center section, a series of opposed segments defining wings extending outwardly from said center section;
said center section and said segments each being formed with at least one cutting edge, said cutting edges being disposed above and opening in said blank to permit discharge of material being removed from said boney material.

23. A cutter blade for use as an element of a disposable acetabular reamer;
said cutter blade being formed from a blank to provide a center section, said center section terminating in transverse end strips;
fingers facing inwardly from the inner edge of said transverse strips;
cutter teeth being formed on said center section and said fingers and tabs being formed on said center section and said fingers to permit attachment of said cutter blade to a support.

24. The cutter blade of claim 23, including a buffer of plastic material bonded about the perimeter of said cutter blade to thereby secure
said cutter blade in an arcuate configuration.

25. A method of manufacturing cutter blades for use as an element of acetabular reamers, comprising the steps of:
creating a series of cutter blade profiles in a sheet of flat material;
each said cutter blade profile being formed with a series of cutting edges; openings being formed immediately below said cutting edges;
said cutting edges being deformed upwardly to protrude above the plane of said cutter blade profile;
said cutter blade being molded into a plastic support so as to be held in an arcuate shape by said support.

26. The method of manufacturing cutter blades of claim 25, wherein said profile, said cutting edges and said openings being formed by a photochemical etching process.

27. The method of manufacturing cutter blades from a flat blank for use as an element in acetabular reamers consisting of the forming of one or more cutting teeth while the blade, or blades are generally flat, and, thereafter, shaping said cutter blade.

28. The method of manufacturing cutter blades for use as an element in acetabular reamers consisting of sharpening of one or more cutting teeth while the blade, or blades, are generally flat.

29. The method of manufacturing cutter blades for use as an element in acetabular reamers consisting of elevating the edge of one or more cutting teeth while the blade, or blades, are generally flat.

30. The method of claim 27, whereby the blade is joined to a support such that when rotated it cuts a generally hemispherical shape.

31. The method of claim 28, whereby the blade is joined to a support such that when rotated it cuts a generally hemispherical shape.

32. The method of claim 29, whereby the blade is joined to a support such that when rotated it cuts a generally hemispherical shape.

33. An acetabular reamer wherein the cutter blade is joined to a support such that when rotated it cuts a generally hemispherical shape.

34. An acetabular reamer wherein the one or more cutter blades is comprised of serially spaced segments to allow the segments of the cutter blade to bend into a generally hemispherical surface.

35. An acetabular reamer wherein the metallic blank is heat treated prior to being formed into a generally hemispherical shape.
